(19) United States
(12) Patent Application Publication
(10) Pub. No.: US 2019/0093002 A1
(43) Pub. Date: Mar. 28, 2019

(54) HYDROCARBON RECOVERY COMPOSITION AND A METHOD FOR USE THEREOF

(71) Applicant: Shell Oil Company, Houston, TX (US)

(72) Inventors: Michael Joseph Doll, Houston, TX (US); Lori Ann Crom, Houston, TX (US); David Perez-Regalado, Amsterdam (NL)

(21) Appl. No.: 16/197,489

(22) Filed: Nov. 21, 2018

(51) Int. Cl. C09K 8/584; C07C 303/14

(52) U.S. Cl. CPC C09K 8/584; C07C 303/14

(57) ABSTRACT

The invention relates to process for preparing an internal olefin sulfonate, comprising: a) sulfonating an internal olefin to produce a first sulfonated internal olefin mixture; and b) contacting the first sulfonated internal olefin mixture with water, caustic and a non-ionic surfactant to form a neutralized sulfonated internal olefin mixture wherein greater than 10 wt. % of the non-ionic surfactant is added.

### FIELD OF THE INVENTION

The present invention relates to a hydrocarbon recovery composition, a method of preparing the hydrocarbon recovery composition and a method of recovering hydrocarbons from a hydrocarbon formation.

### BACKGROUND OF THE INVENTION

Hydrocarbons, such as crude oil, may be recovered from hydrocarbon containing formations (or reservoirs) by penetrating the formation with one or more wells, which may allow the hydrocarbons to flow to the surface. A hydrocarbon containing formation may have one or more natural components that may aid in mobilising hydrocarbons to the surface of the wells. For example, gas may be present in the formation at sufficient levels to exert pressure on the hydrocarbons to mobilise them to the surface of the production wells. These are examples of so-called “primary oil recovery”.

However, reservoir conditions (for example permeability, hydrocarbon concentration, porosity, temperature, pressure, composition of the rock, concentration of divalent cations (or hardness), etc.) can significantly impact the economic viability of hydrocarbon production from any particular hydrocarbon containing formation.

Furthermore, the above-mentioned natural pressure-providing components may become depleted over time, often long before the majority of hydrocarbons have been extracted from the reservoir. Therefore, supplemental recovery processes may be required and used to continue the recovery of hydrocarbons, such as oil, from the hydrocarbon containing formation. Such supplemental oil recovery is often called “secondary oil recovery” or “tertiary oil recovery”. Examples of known supplemental processes include waterflooding, polymer flooding, gas flooding, alkali flooding, thermal processes, solution flooding, solvent flooding, or combinations thereof. Various surfactants may be used in these supplemental processes, but some surfactants are less effective under certain reservoir conditions.

In recent years there has been increased activity in developing new and improved methods of chemical Enhanced Oil Recovery (cEOR) for maximising the yield of hydrocarbons from a subterranean reservoir. In surfactant cEOR the mobilisation of residual oil saturation is achieved through surfactants which generate a sufficiently (ultra) low crude oil/water interfacial tension (IFT) to give a capillary number large enough to overcome capillary forces and allow the oil to flow. However, different reservoirs can have very different characteristics (for example crude oil type, temperature, water composition—salinity, hardness etc.), and therefore, it is desirable that the structures and properties of the added surfactant(s) be matched to the particular conditions of a reservoir to achieve the required low IFT. In addition, other important criteria must be fulfilled, such as low rock retention or adsorption, compatibility with polymer, thermal and hydrolytic stability and acceptable cost (including ease of commercial scale manufacture).

Compositions and methods for cEOR utilising an internal olefin sulfonate (IOS) as surfactant are described in U.S. Pat. Nos. 4,597,879, 4,979,564, and 5,068,043. Surfactants for enhanced hydrocarbon recovery are normally provided to the hydrocarbon containing formation by admixing it with water and/or brine which may originate from the formation from which hydrocarbons are to be recovered, thereby forming a fluid that can be injected into the hydrocarbon containing formation. The surfactant amount in such injectable water containing fluid is generally in the range of from 0.1 to 1 wt. %.

The process for preparing the IOS has a significant impact on the physical properties of the IOS and its performance as a cEOR surfactant. An improved process for preparing the IOS, especially a high active matter IOS is very desirable.

### SUMMARY OF THE INVENTION

The invention provides a process for preparing an internal olefin sulfonate, comprising: a) sulfonating an internal olefin to produce a first sulfonated internal olefin mixture; and b) contacting the first sulfonated internal olefin mixture with water, caustic and a non-ionic surfactant to form a neutralized sulfonated internal olefin mixture wherein greater than 10 wt. % of the non-ionic surfactant is added.

The invention further provides a method of treating a hydrocarbon containing formation comprising providing a hydrocarbon recovery Composition to at least a portion of the hydrocarbon containing formation and allowing the hydrocarbon recovery composition to contact the formation wherein the hydrocarbon recovery composition is prepared by a) sulfonating an internal olefin to produce a first sulfonated internal olefin mixture; and b) contacting the first sulfonated internal olefin mixture with water, caustic and a non-ionic surfactant to form a neutralized sulfonated internal olefin mixture wherein greater than 10 wt. % of the non-ionic surfactant is added.

### DETAILED DESCRIPTION OF THE INVENTION

The present invention relates to a hydrocarbon recovery composition comprising one or more internal olefin sulfonates. In one embodiment, the hydrocarbon recovery composition comprises a mixture of internal olefin sulfonates.

The hydrocarbon recovery composition preferably contains water. The active matter content of the aqueous hydrocarbon recovery composition is preferably at least 20 wt. %, more preferably at least 40 wt. %, more preferably at least 50 wt. %, most preferably at least 60 wt. %. In some embodiments, the aqueous hydrocarbon recovery composition may have an even higher active matter, of at least 65 wt. %, at least 70 wt. % or at least 80 wt. %. “Active matter” herein means the total of anionic species in the aqueous composition, but excluding any inorganic anionic species, for example, sodium sulfate. The active matter content concerns the active matter content of the hydrocarbon recovery composition before it may be combined with a hydrocarbon removal fluid, which fluid may comprise water (e.g. a brine), to produce an injectable fluid, which injectable fluid may be injected into a hydrocarbon containing formation.

In general, stability of the hydrocarbon recovery composition components at a high temperature is relevant to prevent the components from being decomposed (for example hydrolyzed) at such high temperature. Internal olefin sulfonates (IOS) are known to be heat stable at temperatures of 60° C. or higher. However, in addition to being heat stable, a hydrocarbon recovery composition may also have to withstand a relatively high concentration of divalent cations. The high concentration of divalent cations may have the effect of precipitating the hydrocarbon recovery composition components out of solution. The hydrocarbon recovery composition should have an adequate aqueous solubility as that improves the injectability of the fluid comprising the hydrocarbon recovery composition to be injected into the hydrocarbon containing formation. Further, an adequate aqueous solubility reduces loss of the components through adsorption to rock or surfactant retention as trapped, viscous phases within the hydrocarbon containing formation. Precipitated solutions would not be suitable as they could result in formation plugging.

### Internal Olefin Sulfonate

The hydrocarbon recovery composition comprises an internal olefin sulfonate which comprises internal olefin sulfonate molecules. An internal olefin sulfonate molecule is an alkene or hydroxyalkane which contains one or more sulfonate groups. Examples of such internal olefin sulfonate molecules are hydroxy alkane sulfonates (HAS) and alkene sulfonates (OS).

The internal olefin sulfonate (IOS) is prepared from an internal olefin by sulfonation. An internal olefin and an IOS comprise a mixture of internal olefin molecules and a mixture of IOS molecules, respectively. The molecules differ from each other, for example, in terms of carbon number and/or branching degree.

Branched IOS molecules are IOS molecules derived from internal olefin molecules which comprise one or more branches. Linear IOS molecules are IOS molecules derived from internal olefin molecules which are linear. An internal olefin may be a mixture of linear internal olefin molecules and branched internal olefin molecules. Analogously, an IOS may be a mixture of linear IOS molecules and branched IOS molecules. An internal olefin or IOS may be characterized by its carbon number and/or linearity.

An internal olefin or internal olefin sulfonate mixture may be characterized by its average carbon number. The average carbon number is determined by multiplying the number of carbon atoms of each molecule by the weight fraction of that molecule and then adding the products, resulting in a weight average carbon number. The average carbon number may be determined by gas chromatography (GC) analysis of the internal olefin.

Linearity is determined by dividing the weight of linear molecules by the total weight of branched, linear and cyclic molecules. Substituents (like the sulfonate group and optional hydroxy group in the internal olefin sulfonates) on the carbon chain are not seen as branches. The linearity may be determined by gas chromatography (GC) analysis of the internal olefin.

Within the present specification, “branching index” (BI) refers to the average number of branches per molecule, which may be determined by dividing the total number of branches by the total number of molecules. The branching index may be determined by ¹H-NMR analysis.

When the branching index is determined by ¹H-NMR analysis, the total number of branches equals: [total number of branches on olefinic carbon atoms (olefinic branches)]+[total number of branches on aliphatic carbon atoms (aliphatic branches)]. The total number of aliphatic branches equals the number of methine groups, which latter groups are of formula R₃CH wherein R is an alkyl group. Further, the total number of olefinic branches equals: [number of trisubstituted double bonds]+[number of vinylidene double bonds]+2*[number of tetrasubstituted double bonds]. Formulas for the trisubstituted double bond, vinylidene double bond and tetrasubstituted double bond are shown below. In all of the below formulas, R is an alkyl group.

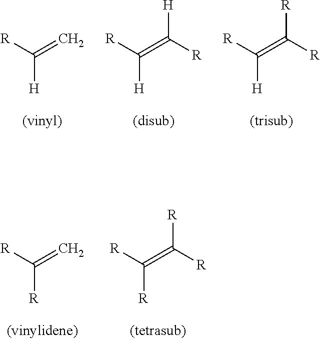

The average molecular weight is determined by multiplying the molecular weight of each surfactant molecule by the weight fraction of that molecule and then adding the products, resulting in a weight average molecular weight.

The hydrocarbon recovery composition comprises an internal olefin sulfonate (IOS) that is at least 40 wt. % linear, more preferably at least 50 wt. %, more preferably at least 60 wt. %, more preferably at least 70 wt. %, more preferably at least 80 wt. %, most preferably at least 90 wt. % linear. For example, 40 to 100 wt. %, more suitably 50 to 100 wt. %, more suitably 60 to 100 wt. %, more suitably 70 to 99 wt. %, most suitably 80 to 99 wt. % of the IOS may be linear. Branches in the IOS may include methyl, ethyl and/or higher molecular weight branches including propyl branches.

Preferably, the IOS is not substituted by groups other than sulfonate groups and optionally hydroxy groups. The IOS preferably has an average carbon number in the range of from 5 to 40, more preferably 10 to 35, more preferably 15 to 30, most preferably 17 to 28.

In one embodiment the IOS may be selected from the group consisting of C₁₅₋₁₈ IOS, C₁₉₋₂₃ IOS, C_{20 24} IOS, C_{24 28} IOS and mixtures thereof, wherein “IOS” stands for “internal olefin sulfonate”. Suitable internal olefin sulfonates include those from the ENORDET™ O series of surfactants commercially available from Shell Chemical.

“C₁₅₋₁₈ internal olefin sulfonate” (C₁₅₋₁₈ IOS) as used herein means a mixture of internal olefin sulfonate molecules wherein the mixture has an average carbon number of from 16 to 17 and at least 50% by weight, preferably at least 65% by weight, more preferably at least 75% by weight, most preferably at least 90% by weight, of the internal olefin sulfonate molecules in the mixture contain from 15 to 18 carbon atoms.

“C₁₉₋₂₃ internal olefin sulfonate” (C₁₉₋₂₃ IOS) as used herein means a mixture of internal olefin sulfonate molecules wherein the mixture has an average carbon number of from 21 to 23 and at least 50% by weight, preferably at least 60% by weight, of the internal olefin sulfonate molecules in the mixture contain from 19 to 23 carbon atoms.

“C₂₀₋₂₄ internal olefin sulfonate” (C₂₀₋₂₄ IOS) as used herein means a mixture of internal olefin sulfonate molecules wherein the mixture has an average carbon number of from 20 to 23 and at least 50% by weight, preferably at least 65% by weight, more preferably at least 75% by weight, most preferably at least 90% by weight, of the internal olefin sulfonate molecules in the mixture contain from 20 to 24 carbon atoms.

“C₂₄₋₂₈ internal olefin sulfonate” (C₂₄₋₂₈ IOS) as used herein means a mixture of internal olefin sulfonate molecules wherein the mixture has an average carbon number of from 24.5 to 27 and at least 40% by weight, preferably at least 45% by weight, of the internal olefin sulfonate molecules in the mixture contain from 24 to 28 carbon atoms.

Further, for the internal olefin sulfonates which are substituted by sulfonate groups, the cation may be any cation, such as an ammonium, alkali metal or alkaline earth metal cation, preferably an ammonium or alkali metal cation.

### IOS Production Method

An IOS molecule is made from an internal olefin molecule whose double bond is located anywhere along the carbon chain except at a terminal carbon atom. Internal olefin molecules may be made by double bond isomerization of alpha olefin molecules whose double bond is located at a terminal position. Generally, such isomerization results in a mixture of internal olefin molecules whose double bonds are located at different internal positions. The distribution of the double bond positions is mostly thermodynamically determined. Further, that mixture may also comprise a minor amount of non-isomerized alpha olefins. Still further, because the starting alpha olefin may comprise a minor amount of paraffins (non-olefinic alkanes), the mixture resulting from alpha olefin isomeration may likewise comprise that minor amount of unreacted paraffins.

The amount of alpha olefins in the internal olefin may be up to 5%, for example 0 to 4 wt. % based on total composition. Further, the amount of paraffins in the internal olefin may be up to 2 wt. %, for example up to 1 wt. % based on total composition.

Suitable processes for making an internal olefin include those described in U.S. Pat. Nos. 5,510,306; 5,633,422; 5,648,584; 5,648,585; 5,849,960; and EP 0830315.

In the sulfonation step, the internal olefin is contacted with a sulfonating agent. The reaction of the sulfonating agent with an internal olefin leads to the formation of cyclic intermediates known as beta-sultones, which can undergo isomerization to unsaturated sulfonic acids and the more stable gamma- and delta-sultones. The sulfonating agent may be sulfur trioxide (SO₃), sulfuric acid or oleum. The mole ratio of sulfonating agent to internal olefin may be 0.5:1 to 2:1, preferably 0.8:1 to 1.8:1, more preferably 1:1 to 1.7:1, and most preferably 1:1 to 1.6:1.

If sulfur trioxide is used as the sulfonating agent, the sulfur trioxide may be provided as a gas stream comprising a carrier gas and sulfur trioxide. The carrier gas may be air or an inert gas, for example, nitrogen. The concentration of sulfur trioxide in the gas stream may be from 0.5 to 10 vol. %, preferably from 1 to 8 vol. %, more preferably from 2 to 7 vol. % based on the volume of the carrier gas.

The sulfonation step with SO₃ is preferably carried out in a film reactor, for example a “falling-film reactor”, where the olefin feed is continuously fed onto the inside surfaces of a tube and gaseous SO₃ is fed into the tube to react with the (falling) olefin film in a controlled manner. The reactor may be cooled with a cooling means, which is preferably water, having a temperature preferably not exceeding 90° C., especially a temperature in the range of from 10 to 70° C., more suitably 20 to 60° C., most suitably 20 to 55° C., for example by flowing the cooling means at the outside walls of the reactor. The desired temperature for the cooling means may depend on the molecular weight and pour point of the feed to and of the reaction mixture in the sulfonation reactor. The sulfonation step may be carried out batchwise, semi-continuously or continuously, preferably continuously.

In a next step, sulfonated internal olefin from the sulfonation step is contacted with a base-containing solution in a neutralization step. In this step, beta-sultones are converted into beta-hydroxyalkane sulfonates, whereas gamma- and delta-sultones are converted into gamma-hydroxyalkane sulfonates and delta-hydroxyalkane sulfonates, respectively. A portion of the hydroxyalkane sulfonates may be dehydrated into alkene sulfonates.

The sulfonated internal olefin is preferably subjected to the neutralization step directly after it is formed, without removing any of the molecules formed in the sulfonation step. In some embodiments, there may be an intermediate step between the sulfonation step and the neutralization step. For example, an aging step may be performed where the sulfonated internal olefins can age for a certain time period.

The base-containing solution comprises a base and a solvent. The base may be a water-soluble base, which may be selected from the group consisting of hydroxides, carbonates and bicarbonates of an alkali metal ion, for example, sodium or potassium, of an alkaline earth metal ion, or of an ammonium ion, and amine compounds. Examples of bases include sodium hydroxide and sodium carbonate. The solvent for the base is preferably water.

The neutralization step is preferably conducted with an excessive molar amount of the base. If the final IOS product is acidic then it can cause corrosion of process equipment or decomposition of the IOS. In a preferred embodiment, the IOS product contains a residual amount of base, for example, from 0.1 to 2 wt. % based on the active matter of the IOS. The amount of base that is fed to the neutralization step may be added such that the molar ratio of base fed to the neutralization step to sulfonating agent fed to the sulfonation step is higher than 1, preferably from 1 to 1.4, more preferably from 1.1 to 1.3.

The temperature of the neutralization step may vary within wide ranges, for example, from 0 to 250° C. The neutralization step is preferably carried out at a temperature in the range of from 0 to 100° C., more preferably 10 to 95° C., more preferably 20 to 90° C., most preferably 30 to 85° C. The time of the neutralization step may also vary within wide ranges, for example, from 5 minutes to 4 hours. The neutralization step may be carried out batchwise, semi-continuously or continuously. The neutralization step may be carried out in a continuously stirred tank reactor or a plug flow reactor.

The neutralization step is carried out in the presence of a non-ionic surfactant, for example through adding the non-ionic surfactant before or during the contacting of sulfonated internal olefin with the base containing solution. When the non-ionic surfactant is added before the contacting of sulfonated internal olefin with the base containing solution, it may be added to the sulfonated internal olefin or to the base containing solution or to both.

By contacting the sulfonated internal olefin with the base containing solution in the presence of a non-ionic surfactant, the IOS produced has improved physical characteristics. For example, the mobility of the reaction mixture is advantageously high for it to be handled easily in terms of storage, pumping and mass transfer. An additional advantage of that is that solutions comprising the internal olefin sulfonate and the non-ionic surfactant can be prepared wherein the concentration of the IOS is relatively high (high active matter) as compared to the situation wherein no non-ionic surfactant would be used. Preferred embodiments of suitable non-ionic surfactants are further described herein.

As mentioned, the non-ionic surfactant added to the neutralization step increases mobility which results in more intimate mixing of the sulfonated internal olefin with the base-containing solution. This improved mixing improves mass transfer and promotes the desirable reaction of the sultones and alkene sulfonic acids with the base and limits the reverse reaction of beta-sultones into internal olefins and SO₃. In addition, the added non-ionic surfactant serves as an important component of the mixture when used for cEOR.

The non-ionic surfactant is added to or present in the neutralization step such that the amount of non-ionic surfactant is greater than 5 wt. %, based on the weight of the active matter of IOS. The non-ionic surfactant is preferably present in an amount of greater than 10 wt. %, preferably at least 15 wt. %. The non-ionic surfactant may be present in an amount of from 11 to 20 wt. %, preferably in an amount of 12 to 18 wt. %.

The neutralization step is followed by a hydrolysis step. In the hydrolysis step, the product from the neutralization step is further reacted through conversion into sulfonate compounds. The hydrolysis step is therefore preferably carried out at an elevated temperature, for example in order to convert sultones, especially delta-sultones, into active matter. Preferably, the temperature in the hydrolysis step is higher than the temperature in the neutralization step. Preferably, the temperature in the hydrolysis step is from 90 to 250° C., more preferably 95 to 220° C., more preferably 100 to 190° C., most preferably 140 to 180° C. The hydrolysis time may be 5 minutes to 4 hours.

Preferably, the product from the neutralization step is directly, without extracting unreacted internal olefin molecules and without removing the base and solvent, subjected to hydrolysis.

The hydrolysis step may be carried out batchwise or continuously, preferably continuously. The hydrolysis step is preferably carried out in a plug flow reactor.

U.S. Pat. Nos. 4,183,867, 4,248,793 and EP0351928A1, the disclosures of all of which are incorporated herein by reference, disclose processes which can be used to make internal olefin sulfonates in the process of the present invention.

An IOS comprises a range of different molecules, which may differ from one another in terms of carbon number, being branched or unbranched, number of branches, molecular weight and number and distribution of functional groups such as sulfonate and hydroxyl groups. An IOS comprises both hydroxyalkane sulfonate molecules and alkene sulfonate molecules and possibly also di-sulfonate molecules. Di-sulfonate molecules originate from a further sulfonation of for example an alkene sulfonic acid.

The IOS may comprise at least 30% hydroxyalkane sulfonate molecules, up to 70% alkene sulfonate molecules and up to 15% di-sulfonate molecules. Suitably, the IOS comprises from 40% to 95% hydroxyalkane sulfonate molecules, from 5% to 50% alkene sulfonate molecules and from 0% to 10% di-sulfonate molecules. Beneficially, the IOS comprises from 50% to 90% hydroxyalkane sulfonate molecules, from 10% to 40% alkene sulfonate molecules and from less than 1% to 5% di-sulfonate molecules. More beneficially, the IOS comprises from 70% to 90% hydroxyalkane sulfonate molecules, from 10% to 30% alkene sulfonate molecules and less than 1% di-sulfonate molecules. The composition of the IOS may be measured using a mass spectrometry technique.

### Non-Ionic Surfactant

The non-ionic surfactant that is added to the IOS during the neutralization step may be an alkoxylated alcohol which is a compound of the formula (I)

R—O—[PO]ₓ[EO]_{y} Formula (I)

wherein R is a hydrocarbyl group, PO is a propylene oxide group, EO is an ethylene oxide group, x is the number of propylene oxide groups, and y is the number of ethylene oxide groups.

The hydrocarbyl group R in formula (I) is preferably aliphatic. When the hydrocarbyl group R is aliphatic, it may be an alkyl group, cycloalkyl group or alkenyl group, suitably an alkyl group. The hydrocarbyl group is preferably an alkyl group. The hydrocarbyl group may be substituted by another hydrocarbyl group as described hereinbefore or by a sub stituent which contains one or more heteroatoms, such as a hydroxy group or an alkoxy group.

The non-alkoxylated alcohol R—OH, from which the hydrocarbyl group R in the above formula (I) originates, may be an alcohol containing 1 hydroxyl group (mono-alcohol) or an alcohol containing of from 2 to 6 hydroxyl groups (poly-alcohol). Suitable examples of poly-alcohols are diethylene glycol, dipropylene glycol, glycerol, pentaerythritol, trimethylolpropane, sorbitol and mannitol. The hydrocarbyl group R in the above formula (I) preferably originates from a non-alkoxylated alcohol R—OH which only contains 1 hydroxyl group (mono-alcohol). Further, the alcohol may be a primary or secondary alcohol, preferably a primary alcohol.

The non-alkoxylated alcohol R—OH, wherein R is an aliphatic group and from which the hydrocarbyl group R in the above formula (I) originates, may comprise a range of different molecules which may differ from one another in terms of carbon number for the aliphatic group R, the aliphatic group R being branched or unbranched, the number of branches for the aliphatic group R, and the molecular weight. Generally, the hydrocarbyl group R may be a branched hydrocarbyl group or an unbranched (linear) hydrocarbyl group. Further, the hydrocarbyl group R may be a branched hydrocarbyl group which has a branching index equal to or greater than 0.3.

The hydrocarbyl group R in the above formula (I) is preferably an alkyl group. The alkyl group has a weight average carbon number within a wide range, namely 5 to 32, more suitably 6 to 25, more suitably 7 to 22, more suitably 8 to 20, most suitably 9 to 17. In a case where the alkyl group contains 3 or more carbon atoms, the alkyl group is attached either via its terminal carbon atom or an internal carbon atom to the oxygen atom, preferably via its terminal carbon atom. Further, the weight average carbon number of the alkyl group is at least 5, preferably at least 6, more preferably at least 7, more preferably at least 8, more preferably at least 9, more preferably at least 10, more preferably at least 11, most preferably at least 12. Still further, the weight average carbon number of the alkyl group is at most 32, preferably at most 25, more preferably at most 20, more preferably at most 17, more preferably at most 16, more preferably at most 15, more preferably at most 14, most preferably at most 13.

Further, the alkyl group R in the above formula (I) is preferably a branched alkyl group which has a branching index equal to or greater than 0.3. The branching index of the alkyl group R in the above formula (I) is preferably of from 0.3 to 3.0, most preferably 1.2 to 1.4. Further, the branching index is at least 0.3, preferably at least 0.5, more preferably at least 0.7, more preferably at least 0.9, more preferably at least 1.0, more preferably at least 1.1, most preferably at least 1.2. Still further, the branching index is preferably at most 3.0, more preferably at most 2.5, more preferably at most 2.2, more preferably at most 2.0, more preferably at most 1.8, more preferably at most 1.6, most preferably at most 1.4.

The alkylene oxide groups in the above formula (I) comprise ethylene oxide (EO) groups or propylene oxide (PO) groups or a mixture of ethylene oxide and propylene oxide groups. In addition, other alkylene oxide groups may be present, such as butylene oxide groups. Preferably, the alkylene oxide groups consist of ethylene oxide groups or propylene oxide groups or a mixture of ethylene oxide and propylene oxide groups. In case of a mixture of different alkylene oxide groups, the mixture may be random or blockwise, preferably blockwise. In the case of a blockwise mixture of ethylene oxide and propylene oxide groups, the mixture preferably contains one EO block and one PO block, wherein the PO block is attached via an oxygen atom to the hydrocarbyl group R.

In the above formula (I), x is the number of propylene oxide groups and is of from 0 to 80. The average value for x is of from 0.5 to 80, preferably of from 3 to 20, and more preferably from 4 to 15. The average number of propylene oxide groups is referred to as the average PO number.

Further, in the above formula (I), y is the number of ethylene oxide groups and is of from 0 to 60. The average value for y is of from 0.5 to 80, preferably of from 3 to 20, and more preferably from 4 to 15. The average number of ethylene oxide groups is referred to as the average EO number

In the above formula (I), y may be 0, in which case the alkylene oxide groups in the above formula (I) comprise PO groups but no EO groups. In the latter case, the average value for the sum of x and y equals the above-described average value for x.

In the above formula (I), x may be 0, in which case the alkylene oxide groups in the above formula (I) comprise EO groups but no PO groups. In the latter case, the average value for the sum of x and y equals the above-described average value for y.

Further, in the above formula (I), each of x and y may be at least 1, in which case the alkylene oxide groups in the above formula (I) comprise PO and EO groups. In the latter case, the average value for the sum of x and y may be of from 1 to 80, suitably of from 3 to 20, and more suitably of from 4 to 15.

### Non-Ionic Surfactant Production Method

The non-alkoxylated alcohol R-OH, from which the hydrocarbyl group R in the above formula (I) originates, may be prepared in any way. For example, a primary aliphatic alcohol may be prepared by hydroformylation of a branched olefin. Preparations of branched olefins are described in U.S. Pat. Nos. 5,510,306; 5,648,584 and 5,648,585. Preparations of branched long chain aliphatic alcohols are described in U.S. Pat. Nos. 5,849,960; 6,150,222; 6,222,077. In another embodiment, the hydrocarbyl group in the alcohol is linear.

### Alkoxylation

The above-mentioned (non-alkoxylated) alcohol R-OH, from which the hydrocarbyl group R in the above formula (I) originates, may be alkoxylated by reacting with alkylene oxide in the presence of an appropriate alkoxylation catalyst. The alkoxylation catalyst may be potassium hydroxide or sodium hydroxide which are commonly used commercially. Alternatively, a double metal cyanide catalyst may be used, as described in U.S. Pat. No. 6,977,236. Still further, a lanthanum-based or a rare-earth metal-based alkoxylation catalyst may be used, as described in U.S. Pat. Nos. 5,059,719 and 5,057,627. The alkoxylation reaction temperature may range from 90° C. to 250° C., suitably 120 to 220° C., and super atmospheric pressures may be used if it is desired to maintain the alcohol substantially in the liquid state.

Preferably, the alkoxylation catalyst is a basic catalyst, such as a metal hydroxide, which catalyst contains a Group IA or Group IIA metal ion. Suitably, when the metal ion is a Group IA metal ion, it is a lithium, sodium, potassium or cesium ion, more suitably a sodium or potassium ion, most suitably a potassium ion. Suitably, when the metal ion is a Group IIA metal ion, it is a magnesium, calcium or barium ion. Thus, suitable examples of the alkoxylation catalyst are lithium hydroxide, sodium hydroxide, potassium hydroxide, cesium hydroxide, magnesium hydroxide, calcium hydroxide and barium hydroxide, more suitably sodium hydroxide and potassium hydroxide, most suitably potassium hydroxide. Usually, the amount of such alkoxylation catalyst is of from 0.01 to 5 wt. %, more suitably 0.05 to 1 wt. %, most suitably 0.1 to 0.5 wt. %, based on the total weight of the catalyst, alcohol and alkylene oxide (i.e. the total weight of the final reaction mixture).

The alkoxylation procedure serves to introduce a desired average number of alkylene oxide units per mole of alcohol alkoxylate (that is alkoxylated alcohol), wherein different numbers of alkylene oxide units are distributed over the alcohol alkoxylate molecules. For example, treatment of an alcohol with 7 moles of alkylene oxide per mole of primary alcohol results in the alkoxylation of each alcohol molecule with an average of 7 alkylene oxide groups, although a substantial proportion of the alcohol will have become combined with more than 7 alkylene oxide groups and an approximately equal proportion will have become combined with less than 7. In a typical alkoxylation product mixture, there may also be a minor proportion of unreacted alcohol.

Non-alkoxylated alcohols from which the hydrocarbyl group R in the above formula (I) originates are commercially available. Suitable examples of a commercially available alcohol mixture are NEODOL™ 91 (a mixture of C9, C10 and C11 alcohols), NEODOL™ 45 (a mixture of C14 and C15 alcohols) and NEODOL™ 25 (a mixture of C12, C13, C14 and C125 alcohols). These alcohols may be ethoxylated to form the non-ionic surfactant and commercially available ethoxylated alcohols that are suitable examples are NEODOL™ 91-8 (where the average number of EO groups is 8), NEODOL™ 45-7 (where the average number of EO groups is 7) and NEODOL™ 25-12 (where the average number of EO groups is 12).

In another embodiment, non-alkoxylated alcohols R—OH, from which the hydrocarbyl group R in the above formula (I) for the alkoxylated alcohol and/or alkoxylated alcohol derivative originates, wherein R is a branched alkyl group which has a branching index equal to or greater than 0.3 and which has a weight average carbon number of from 5 to 32, are commercially available. A suitable example of a commercially available alcohol mixture is NEODOL™ 67, which includes a mixture of C₁₆ and C₁₇ alcohols of the formula R—OH, wherein R is a branched alkyl group having a branching index of about 1.3, sold by Shell Chemical LP. Shell Chemical LP also manufactures a C₁₂/C₁₃ analogue alcohol of NEODOL™ 67, which includes a mixture of C₁₂ and C₁₃ alcohols of the formula R—OH, wherein R is a branched alkyl group having a branching index of about 1.3, and which is used to manufacture alcohol alkoxy sulfate (AAS) products branded and sold as ENORDET™ enhanced oil recovery surfactants. Another suitable example is EXXAL™ 13 tridecylalcohol (TDA), sold by ExxonMobil, which is of the formula R—OH wherein R is a branched alkyl group having a branching index of about 2.9 and having a carbon number distribution wherein 30 wt. % is C₁₂, 65 wt. % is C₁₃ and 5 wt. % is C₁₄. Yet another suitable example is MARLIPAL® tridecylalcohol (TDA), sold by Sasol, which product is of the formula R—OH wherein R is a branched alkyl group having a branching index of about 2.2 and having 13 carbon atoms.

### Cosolvent

A cosolvent (or solubilizer) may be added to increase the solubility of the surfactants in the hydrocarbon recovery composition and/or in the below-mentioned injectable fluid comprising the composition. Suitable examples of cosolvents are polar cosolvents, including lower alcohols (for example sec-butanol, isopropyl alcohol and tert-amyl alcohol) and polyethylene glycol. Any amount of cosolvent needed to dissolve the surfactant at a certain salt concentration (salinity) may be easily determined by a skilled person through routine tests.

### Additional Components

A hydrotrope may be added to increase the solubility of the surfactants in the hydrocarbon recovery composition and/or in the below-mentioned injectable fluid comprising the composition. Suitable examples of hydrotropes include both aryl and non-aryl compounds. The aryl compounds are generally aryl sulfonates or short-chain alkyl-aryl sulfonates in the form of their alkali metal salts (for example sodium toluene sulfonate, potassium toluene sulfonate, sodium xylene sulfonate, ammonium xylene sulfonate, potassium xylene sulfonate, calcium xylene sulfonate, sodium cumene sulfonate, and ammonium cumene sulfonate). Suitable examples of non-aryl hydrotropes are sulfonates whose alkyl moiety contains from 1 to 8 carbon atoms (for example butane sulfonate and hexane sulfonate).

Viscosity modifiers other than the above-described non-ionic surfactant of formula (I) may be used in addition to the non-ionic surfactant and be included in the hydrocarbon recovery composition. An embodiment of a viscosity modifier is a linear or branched C₁ to C₆ monoalkylether of mono- or di-ethylene glycol. Suitable examples are diethylene glycol monobutyl ether (DGBE), ethylene glycol monobutyl ether (EGBE) and triethylene glycol monobutyl ether (TGBE). Further, a linear or branched C₁ to C₆ dialkylether of mono-, di- or triethylene glycol, such as ethylene glycol dibutyl ether (EGDE), may be used as a further viscosity modifier.

The hydrocarbon recovery composition may comprise a base (herein also referred to as “alkali”), preferably an aqueous soluble base, including alkali metal containing bases such as for example sodium carbonate and sodium hydroxide.

In addition to the non-ionic surfactant and the internal olefin sulfonate, the hydrocarbon recovery composition may comprise one or more compounds that function as a pH buffer. A pH buffer is an aqueous solution comprising a weak acid and its conjugate base or a weak base and its conjugate acid. The pH of the buffer changes very little when a small amount of a strong acid or base is added to the buffer. pH buffer solutions can be used to keep the pH at a substantially constant value in the hydrocarbon recovery composition.

The pH buffer may comprise a base selected from the group consisting of ammonia, trimethyl ammonia, pyridine and other amine containing compounds and ammonium hydroxide. The pH buffer may comprise an inorganic base. Preferred embodiments of inorganic bases are the conjugate bases of boric acid and phosphoric acid.

The pH buffer may comprise an acid selected from the group consisting of formic acid, acetic acid, propanoic acid, butanoic acid, pentanoic acid, hexanoic acid, heptanoic acid, octanoic acid, nonanoic acid, decanoic acid, trichloroacetic acid, hydrofluoric acid, hydrocyanic acid, phosphoric acid, oxalic acid, nitrous acid, benzoic acid, ascorbic acid, boric acid, chromic acid, citric acid, carbonic acid, lactic acid, sulfurous acid, uric acid. The pH buffer may comprise KH₂PO₄, Na₂HPO₄ or mixtures thereof.

The hydrocarbon recovery composition may additionally comprise an acid which has a pKₐ between 6 and 12 and the conjugate base of such acid. The acid/conjugate base mixture may function as a stabilizing buffer. The acid which has a pKₐ between 6 and 12 and the conjugate base of such acid, and amounts and concentrations of these, may be any one of those as disclosed in US 2016/0177173.

### Hydrocarbon Recovery Method

The hydrocarbon recovery composition may be combined with a hydrocarbon removal fluid to produce an injectable fluid, wherein the hydrocarbon removal fluid 1) comprises water (e.g. a brine) and 2) may comprise divalent cations in any concentration, suitably in a concentration of 100 or more parts per million by weight (ppmw), after which the injectable fluid may be injected into the hydrocarbon containing formation.

The present invention further relates to a method of treating a hydrocarbon containing formation, comprising the following steps:

a) providing a hydrocarbon recovery composition to at least a portion of the formation;

b) allowing the hydrocarbon recovery composition to contact the formation.

Normally, surfactants for enhanced hydrocarbon recovery are transported to a hydrocarbon recovery location and stored at that location in the form of an aqueous composition containing for example 15 to 70 wt. % surfactant. At the hydrocarbon recovery location, the surfactant concentration of such composition would then be further reduced to 0.05-2 wt. %, by diluting the composition with water or brine, before it is injected into a hydrocarbon containing formation. By such dilution with water or brine, an aqueous fluid is formed which fluid can be injected into the hydrocarbon containing formation. Advantageously, a more concentrated aqueous composition having an active matter content of for example 40-70 wt. %, as described above, may be transported to the location and stored there.

The total amount of the surfactants in the injectable fluid may be of from 0.05 to 2 wt. %, preferably 0.1 to 1.5 wt. %, more preferably 0.1 to 1.2 wt. %, most preferably 0.2 to 1.0 wt. %.

### Hydrocarbon Containing Formation

A “hydrocarbon containing formation” is defined as a sub-surface hydrocarbon containing formation.

The hydrocarbon containing formation may be a crude oil-bearing formation. Different crude oil-bearing formations or reservoirs differ from each other in terms of crude oil type. First, the API may differ among different crude oils. Further, different crude oils comprise varying amounts of saturates, aromatics, resins and asphaltenes. The 4 components are commonly abbreviated as “SARA”. Further, crude oils comprise varying amounts of acidic and basic components, including naphthenic acids and basic nitrogen compounds. Still further, crude oils comprise varying amounts of paraffin wax. These components are present in heavy (low API) crude oils and light (high API) crude oils. The overall distribution of such components in a crude oil is a direct result of geochemical processes. The properties of the crude oil in the crude oil-bearing formation may differ widely. For example, in respect of the API and the amounts of the above-mentioned crude oil components comprising saturates, aromatics, resins, asphaltenes, acidic and basic components (including naphthenic acids and basic nitrogen compounds) and paraffin wax, the crude oil may be of one of the types as disclosed in WO 2013030140 and US 2016/0177172.

Hydrocarbons may be produced from hydrocarbon containing formations through wells penetrating such formations. “Hydrocarbons” are generally defined as molecules formed primarily of carbon and hydrogen atoms such as oil and natural gas. Hydrocarbons may also include other elements, such as halogens, metallic elements, nitrogen, oxygen and/or sulfur. Hydrocarbons derived from a hydrocarbon containing formation may include kerogen, bitumen, pyrobitumen, asphaltenes, oils or combinations thereof. Hydrocarbons may be located within or adjacent to mineral matrices within the earth. Matrices may include sedimentary rock, sands, silicilytes, carbonates, diatomites and other porous media.

A “hydrocarbon containing formation” may include one or more hydrocarbon containing layers, one or more non-hydrocarbon containing layers, an overburden and/or an underburden. An overburden and/or an underburden includes one or more different types of impermeable materials. For example, overburden/underburden may include rock, shale, mudstone, or wet/tight carbonate (that is to say an impermeable carbonate without hydrocarbons). For example, an underburden may contain shale or mudstone. In some cases, the overburden/underburden may be somewhat permeable. For example, an underburden may be composed of a permeable mineral such as sandstone or limestone.

Properties of a hydrocarbon containing formation may affect how hydrocarbons flow through an underburden/overburden to one or more production wells. Properties include porosity, permeability, pore size distribution, surface area, salinity or temperature of formation. Overburden/underburden properties in combination with hydrocarbon properties, capillary pressure (static) characteristics and relative permeability (flow) characteristics may affect mobilization of hydrocarbons through the hydrocarbon containing formation.

The hydrocarbon containing formation consists of a pore space and a rock matrix. The pore space of the hydrocarbon containing formation contains an aqueous solution called formation water in addition to hydrocarbon fluids. The rock matrix of the hydrocarbon containing formation or reservoir rock is rich in various elements and compounds. In some embodiments, the rock matrix of the hydrocarbon containing formation can act as a pH buffer.

Two distinctly different types of reservoir rock are generally recognized which are clastic formations and carbonate formations. In Lake, Larry, “Enhanced Oil Recovery”, table 3.3 provides an analysis of eight different rocks, seven clastic (sandstone) samples and one carbonate (limestone) sample. The overview demonstrates that quartz (SiO₂) is the main component of clastic formations and the weight percentage of quartz in these samples varies from 64 to 90%. The remaining components include carbonates, clay minerals and feldspars. Carbonates can be present in the form of calcite, ankerite, dolomite, siderite, and/or other carbonate salts and are a source of multivalent ions in the formation water present in the pore space of the hydrocarbon containing formation. Clay minerals are aluminium silicates with molecular lattices that can contain various mono-valent and divalent ions. An important characteristic of clay minerals is that they have a large surface area and have the ability to exchange cations with the formation water. The formation water is generally in equilibrium with the rock matrix at the time of discovery of the hydrocarbon reservoir; an equilibrium which is established over geological time. For example, formation water may contain Na⁺, K⁺, Ca²⁺, Mg²⁺, Cl⁻, HCO₃⁻ ions and many other trace ions. The presence of bicarbonate ions at a significant level indicates the pH buffering capacity of the hydrocarbon containing formation.

The temperature of the hydrocarbon containing formation may be in a range of from 60 to 150° C. In one embodiment, the temperature of the hydrocarbon containing formation is in the range of from 80 to 120° C.

### EXAMPLES

In this example, seven samples were prepared comprising an IOS mixture. The IOS was prepared in a falling film reactor. During the neutralization step, a non-ionic surfactant was added to the IOS in different amounts. The non-ionic surfactant used in these examples was NEODOL 91-8, an alkoxylated alcohol made from an alcohol mixture comprising C9 to C11 alcohols having an average EO number of 8. The amount of non-ionic surfactant is expressed as the weight percent based on the active matter in the mixture. Table 1 shows the seven samples, the neutralization conditions and the resulting unreacted organic matter (UOM). Samples A, E, F and G were hydrolysed with a hydrolyser residence time of 28 minutes and Samples B, C and D were hydrolysed with a hydrolyser residence time of about 56 minutes.

TABLE 1

| | Non-ionic | Neutralizer | UOM | Active Matter |
|---|---|---|---|---|
| Sample | (wt. %/AM) | temperature (° C.) | (wt. %) | (wt. %) |
| A | 10.4 | 83 | 3.32 | 75.27 |
| B | 15 | 80 | 1.06 | 75.43 |
| C | 10 | 90 | 1.65 | 78.16 |
| D | 10 | 100 | 1.78 | 78.05 |
| E | 15 | 80 | 0.81 | 76.13 |
| F | 10 | 90 | 1.73 | 78.22 |
| G | 10 | 100 | 1.88 | 78.01 |

1. A process for preparing an internal olefin sulfonate, comprising:
a. sulfonating an internal olefin to produce a first sulfonated internal olefin mixture; and
b. contacting the first sulfonated internal olefin mixture with water, caustic and a non-ionic surfactant to form a neutralized sulfonated internal olefin mixture wherein greater than 10 wt. % of the non-ionic surfactant is added.

2. The process of claim 1 wherein the active matter content of the internal olefin sulfonate is 40 to 90 wt. %.

3. The process of any of claims 1-2 wherein the internal olefin has an average carbon number of from 5 to 40.

4. The process of any of claims 1-3 wherein the base is a water-soluble base and the solvent for the base is water.

5. The process of any of claims 1-4 wherein the water-soluble base is selected from the group consisting of hydroxides, carbonates and bicarbonate of an alkali metal ion, of an alkaline earth metal ion, or of an ammonium ion, and amine compounds.

6. The process of any of claims 1-6 wherein the water-soluble base is sodium hydroxide.

7. The process of any of claims 1-6 wherein the non-ionic surfactant is at least 12 wt. % of the mixture.

8. The process of any of claims 1-6 wherein the non-ionic surfactant is at least 15 wt. % of the mixture.

9. The process of any of claims 1-8 wherein the non-ionic surfactant is an alkoxylate of an alcohol having an aliphatic group.

10. The process of claim 9 wherein the non-ionic surfactant is an alcohol ethoxylate of the formula R—O—[R′—O]ₓ—H wherein R is an aliphatic group having from 9 to 11 carbon atoms and x is 8.

11. A method of treating a hydrocarbon containing formation comprising providing a hydrocarbon recovery composition to at least a portion of the hydrocarbon containing formation and allowing the hydrocarbon recovery composition to contact the formation wherein the hydrocarbon recovery composition is prepared by a) sulfonating an internal olefin to produce a first sulfonated internal olefin mixture; and b) contacting the first sulfonated internal olefin mixture with water, caustic and a non-ionic surfactant to form a neutralized sulfonated internal olefin mixture wherein greater than 10 wt. % of the non-ionic surfactant is added.
